(19) United States
(12) Patent Application Publication
(10) Pub. No.: US 2013/0130308 A1
(43) Pub. Date: May 23, 2013

(54) PROCESS FOR DIRECTLY MEASURING MULTIPLE BIODEGRADABILITIES

(71) Applicant: ENVOLURE, Grabels (FR)

(72) Inventors: Nathalie PAUTREMAT, Montpellier (FR); Romy-Alice GOY, Saint Just (FR); Zaynab EL AMRAOUI, Montpellier (FR); Yves DUDAL, Hegenheim (FR)

(73) Assignee: ENVOLURE, Grabels (FR)

(21) Appl. No.: 13/682,830

(22) Filed: Nov. 21, 2012

Related U.S. Application Data
(60) Provisional application No. 61/608,162, filed on Mar. 8, 2012.

(30) Foreign Application Priority Data
Nov. 23, 2011 (FR) 11 60685

(51) Int. Cl. C12Q 1/02

(52) U.S. Cl. CPC C12Q 1/02
USPC 435/34

(57) ABSTRACT

A method for measuring the biodegradability of organic substrates by the fluorescent and/or colorimetric detection of the microbial activity generated by the addition of organic substrates to a mixture of microorganisms.

The present invention relates to a method for the measurement of the biodegradability of organic substrates based on the use of a fluorescent and/or colorimetric bioreagent for assessing the microbial activity generated by the addition of organic substrates to a mixture of microorganisms. This method was developed to allow measurements under either aerobic or anaerobic conditions.

The invention relates to the field of analysis of the biodegradability of organic matter of various types, such as, for example, waters and sludges from wastewater treatment plants, effluents from agribusiness or animal farming, industrial, agribusiness and agricultural wastes or compost.

The organic substrates can be used by the microorganisms as substrates from which they obtain energy and the ability to grow. A number of industrial applications use these biochemical degradation reactions, under aerobic or anaerobic conditions, in particular: wastewater purification, production of high value-added compounds, energy production and agri-food production. The sources of available organic substrates are very varied: crops and crop production residues, food processing residues, domestic and urban wastes, biomass, in particular algal biomass.

Different methods exist for measuring the biodegradability of the organic substrates, i.e. for measuring the extent to which microorganisms will be able to use these substrates in order to convert them. Each field of application uses its own method, very generally based on measuring the product formed during the reaction, sometimes on measuring the degraded substrate. These methods are time-consuming, complex to put in place and require heavy and costly equipment.

Two industrial parameters are more commonly used for measuring the biodegradability of the organic substrates: biochemical (or biological) oxygen demand (BOD) and biochemical methane potential (BMP).

The quantity of organic matter can be assessed by measuring the industrial parameter represented by the biochemical (or biological) oxygen demand. The BOD represents the quantity of dioxygen required by the waterborne aerobic microorganisms to oxidize the organic matter that is dissolved or in suspension in the water. The most commonly used measurement is that of BOD₅. BOD₅ corresponds to the biochemical (or biological) oxygen demand after 5 days' incubation of the sample at a temperature of 20° C. Its standard measurement protocol is described in standard NF EN 1899-1.

The second industrial parameter used is the methane potential which corresponds to the volume of methane produced during the degradation of an organic substrate in the presence of anaerobic bacteria. The usual protocol is based on measuring the volume of methane produced from a known quantity of a sample to be assayed in the presence of a known quantity of anaerobic microorganisms. The assay takes place under conditions (temperature, pH, presence of nutritive elements, etc.) that are favourable to the degradation of the sample. The protocol is described in standard ISO 11734. The methane is then utilized in the form of heat or electricity on site or injected into the municipal networks. The methanization residue, the digestate, can be upcycled in the form of agricultural fertilizer in particular.

International application WO2007/105040 relates to a device for quick estimation of the biochemical oxygen demand of waste drinking water. This device is constituted by an immobilized microbial membrane attached to an electrode, a multimeter and a portable workstation equipped with specially developed software. Measuring the BOD of waste drinking water using this device is more rapid, reproducible and effective as compared to conventional titration-based methods.

Patent EP0855023B1 describes a method for determining the biological oxygen demand of wastewater, in which a wastewater sample is mixed with biologically neutral dilution water to a predetermined degree of dilution. Then, in a biological bath, the oxygen concentration is measured, which is established by virtue of the biological reaction of a predetermined biomass with the diluted wastewater sample in the biological bath. In this method, in discontinuous mode a predetermined quantity of the wastewater sample is added and mixed in the biological bath filled with oxygen-saturated dilution water, and the oxygen consumption is measured, which is established per unit of time, and the biological bath is rinsed with dilution water.

However, these two methods have drawbacks, in particular as they can only be applied to liquid samples, and are quite restrictive to implement.

In order to determine the methane potential, the usual protocol consists of inoculating a certain number of flasks, containing a small quantity of medium to be analyzed, with anaerobic inoculum in the absence of oxygen, often in a nitrogen atmosphere. These flasks are then placed in a waterbath or an incubator, the temperature of which is controlled. By manual analysis of the volume and composition of the emitted gas by chromatography, the volume of methane produced is analyzed periodically, until the cumulative methane volume curve has stabilized. The analysis period is from 20 to 60 days. Thus, this analytical procedure requires costly laboratory equipment and is very complex and demanding in terms of time and work. The biogas and methane composition produced can only be analyzed manually on an ad hoc basis, making it difficult to introduce as a routine within a laboratory. A genuine need therefore exists for an automated assay that can provide good-quality data simply, reliably and quickly.

International application WO 2006/079733 proposes the use of fluorescence to assess the oxidative action of the bacteria on the organic matter to be degraded. A fluorescent probe penetrates into the bacteria and reacts to each electron exchange. This is therefore a direct method of evaluating the bacterial activity. This method only allows assessment of the fate of an organic matter or combinations of the organic matter with certain soil elements and relates essentially to the analysis of liquid samples originating from the soil, but provides no means of obtaining commonly-used industrial parameters, such as the BOD₅ or the BMP.

The article by Dudal Y. et al. (Anal. Bioanal. Chem., (2006), 384, 175-179) includes the content of international application WO 2006/079733 and describes a microplate fluorescence test for measuring the catabolic activity induced by the dissolved organic matter or DOM. This technique makes it possible to calculate, from glucose calibration curves, the mineralizable quantity of a DOM and to classify the DOMs as a function of their capacity to react with bacteria.

The article by Christian Guyard (L'eau, l'industrie et les nuisance, (Jan. 8, 2010), No 334, 51-58) is a review of the different techniques used for the measurement of BOD₅ and the problems inherent in these techniques. Reference is made to the invention which is the subject-matter of international application WO 2006/079733 and to the assumed correlation between the fluorescence measurement and the BOD₅ measurement, without however specifying how it is established.

A purpose of the invention is to overcome the drawbacks of the state of the art, and in particular to improve the following points:

Allowing measurement that is quick and simple to implement;

Proposing a method that is applicable to a number of types of organic samples;

Improving the sensitivity of the method;

Extending the range of concentrations that can be measured by the method;

Proposing a method allowing the use of microorganisms suited to each operational application;

Proposing a method allowing high-throughput working;

Providing results in the form of known industrial parameters.

To this end, the invention proposes to measure the biodegradability of the organic substrates by fluorescent and/or colorimetric detection of the microbial activity generated by the addition of organic substrates to a mixture of microorganisms, to deduce therefrom the expected value for each field of application by calibration and to quantify the organic matter present. This measurement is carried out in a format allowing multiple measurements with automated data processing.

This invention makes it possible to carry out measurements of equivalents using the standardized methods of BOD₅ and of BMP, but the method can also be used for measuring other potentials in the environmental field, such as fermentary or fertilizing potential, and also in the food processing (dairy industry) and petrochemical fields. This method can also be used to carry out the diagnosis of a bioreactor, measuring the purification performances or studying the inhibiting or toxic effects of different products (effluents, organic substrates etc.). It can also make it possible to obtain results which are comparable to those of respirometry.

A subject of the invention is also a method for the direct measurement of the biodegradability of organic samples comprising the following steps:

preparing the sample,

in a microplate, incubating the sample with a fluorescent and/or colorimetric bioreagent and an inoculum of micro-organisms capable of degrading said sample, for a duration comprised between 1 and 48 hours, advantageously between 12 and 48 hours, advantageously between 12 and 24 hours,

analyzing the absorbance—fluorescence emitted by the mixture over time, said absorbance—fluorescence analysis comprising the following two steps:

a) measuring an intensity of absorbance—fluorescence emitted following degradation of the sample by the inoculum of micro-organisms, said fluorescence intensity profile obtained allowing:
either determining the minimum measurement time by analysis of a coefficient of determination of a calibration curve, said calibration curve associating the intensity of the fluorescence with the concentration of organic matter being obtained by carrying out measurements of absorbance—fluorescence on samples of known increasing concentrations constituting a standard range, and/or by comparison with the results obtained on samples of known concentrations by a usual “standardized” method,
or deducing the instantaneous rate of biodegradation profiles,

b) using the intensity of absorbance—fluorescence measurement for calculating a reference organic matter concentration, via a correlation either with a standard range, or with a mathematical model, said method comprising moreover a step of calculating an industrial parameter from the concentration of organic matter calculated in step b).

According to the invention, the instantaneous rate of biodegradation profiles can be calculated by any technique known to a person skilled in the art, in particular by deriving the intensity profile. The rate profiles make it possible, simply by reading, to select the maximum instantaneous rate which is directly correlated with the substrate concentration.

According to the invention, the samples can be sewage, drinking water, industrial effluents, wastewater, organic substrates, in particular from agribusiness or animal farming, wastes from industry, agribusiness or animal farming or compost and sewage sludges, algae, etc.

They can be used directly or after dilution.

In an advantageous embodiment of the invention, the measurement of the intensity of the absorbance—fluorescence is carried out through the underside of the plate.

In another advantageous embodiment of the invention, the sample to be analyzed is taken from a treatment site and the inoculum of micro-organisms comes from a bacterial system, the composition of which is stable over time, that is present on the same site.

If necessary, the inoculum is passed through PES filters with a mesh size of 1.2 μm before use.

According to the invention, the inoculum of microorganisms can originate from the sampling medium itself or be prepared from lyophilized strains or from a culture of bacterial strains. These strains and cultures are commercially available and well known to a person skilled in the art.

According to the invention, when the measurement is carried out under aerobic conditions, it is carried out either with the microplate left open, or covered with a film allowing oxygen exchange without evaporation.

In an advantageous embodiment of the invention, the incubation is carried out for a duration comprised between 1 hour and 24 hours, advantageously between 12 and 24 hours in an aerobic medium and at least 10 hours in an anaerobic medium.

In another embodiment of the invention, the measurements are carried out at least hourly and at most every 15 minutes.

In an advantageous embodiment of the invention, the fluorescence measurement is converted to mgO₂.L⁻¹, the unit of the BOD₅ (biologic oxygen demand on 5 days), by comparison with a standard range.

In another advantageous embodiment of the invention, the fluorescence measurement is converted to mgO₂.L⁻¹, by using a mathematical model associating the fluorescence intensity with the concentration.

In another advantageous embodiment of the invention, the mathematical equation is adjusted according to the fluorescence intensities measured on control solutions of known concentrations placed under the same conditions as the samples to be analyzed.

In another advantageous embodiment of the invention the measurement is carried out under anaerobic conditions, in particular by covering each well of the microplate with paraffin then closing the microplate using a lid, the plate being able to be turned over so that the fluorescence measurement is carried out through the underside.

According to the invention, in the case of a measurement under anaerobic conditions, the measurement time chosen in an automated fashion can be that at which the coefficient of determination of the calibration curve is the closest to 1 over a total duration of incubation.

In an advantageous embodiment of the invention under anaerobic conditions, the fluorescence emitted is converted to LCH₄.Kg⁻¹ of raw matter, according to the usual unit for expressing the methane potential according to a calculation comprising the following steps:

converting the fluorescence intensities to gC-Acetate.Kg⁻¹ using the mathematical equation originating from the linear regression of the calibration curve, or

referring to a database constituted by BMP in LCH₄.Kg⁻¹ of raw matter in order to predict the methane potential in LCH₄.Kg⁻¹ of raw matter.

Advantageously, according to the invention, the methane potential of unknown samples is directly estimated in LCH₄.Kg⁻¹ of raw matter from the calibration curve.

The method according to the invention can utilize a kit comprising at least one microplate, at least one fluorescent and/or colorimetric bioreagent and standard and/or control solutions.

According to the invention, the step of analysis of the absorbance-fluorescence is implemented by a system comprising a fluorescence reader suitable for the microplate format and calculation means arranged for implementing said method.

A subject of the invention is also a method for quantifying organic matter present in a sample implementing the biodegradability measurement described previously.

Thus according to the invention, the measurement comprises several steps:
Setting up the method: choosing the inoculum and determining the optimum incubation time,
Preparing the sample
In a microplate, placing the sample in contact with an inoculum of microorganisms capable of degrading said sample and with a fluorescent and/or colorimetric bioreagent, and incubation,
Analyzing the absorbance and/or fluorescence emitted by the mixture over time.

The inocula originating from different sources, media or environments, under different growth conditions, do not react in the same way when placed in contact with the same substrate. In order to determine the relationship between the bacterial activity measured by absorbance/fluorescence and the industrial parameter aimed for, a characterization by the reference method (BOD or BMP) must be carried out. This characterization is carried out on solutions of the standard ranges used in the method of the invention, either synthetic solutions (example: solution of a mixture of glucose+glutamic acid) or on real samples (example: suspension of ground biodegradable waste at different concentrations). The association between the concentrations of the standard solutions and the industrial parameter is thus defined and is used for the quantification of the samples to be tested.

The minimum and/or optimal incubation time is determined as a function of the data processing method used.

The absorbance—fluorescence analysis takes place in three steps carried out by an automated system:
a) measuring the intensity of the absorbance—fluorescence by a detector, in a minimal measurement time predetermined during the step of characterization of the inoculum,
b) using the intensity profiles or instantaneous rate profiles to calculate a reference organic matter concentration, by means of correlation either with a standard range, or with a mathematical model, this reference molecule being able to be a glucose—glutamic acid mixture for measuring BOD₅ and acetate for measuring BMP or any other molecule representing a model substrate for the biodegradation studied;
c) translating this reference organic matter concentration value into an industrial parameter, from the correlation determined during the characterization of the inoculum.

In certain cases, in particular under anaerobic conditions, steps b) and c) are merged.

The data processing algorithm implemented serves to automate the decision on the reading and incubation time and to interpret absorbance—fluorescence results into biodegradability units commonly used in industry: mgO₂.L⁻¹ for the BOD₅ equivalent conventionally used to assess the biodegradability of wastewater, LCH₄.kg⁻¹ raw matter for the methane potential equivalent.

A subject of the invention is therefore a method for measuring biodegradability, making it possible to obtain an indicative industrial parameter rapidly, simply and automatically, said parameter making it possible to quantify the organic matter present in a sample.

Other features and advantages of the invention will become apparent from the detailed description and from the attached drawings in which:

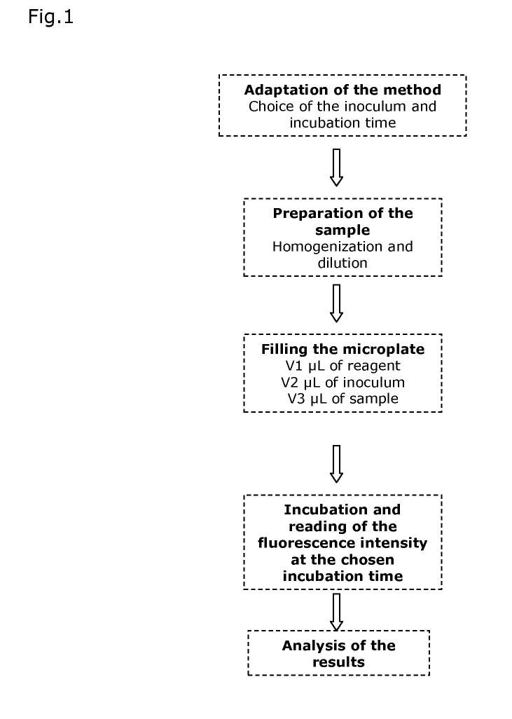

FIG. 1 shows diagrammatically the implementation of the method according to the invention under aerobic conditions;

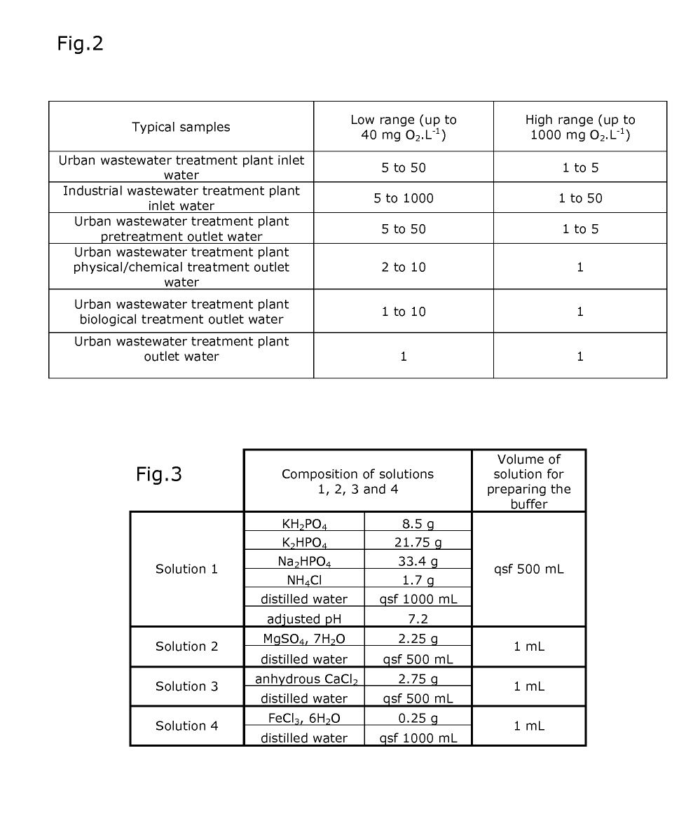

FIG. 2 presents the dilutions of “typical” samples as a function of the range used for measurements under aerobic conditions;

FIG. 3 gives an example of composition of the dilution buffer to be used for preparing the samples.

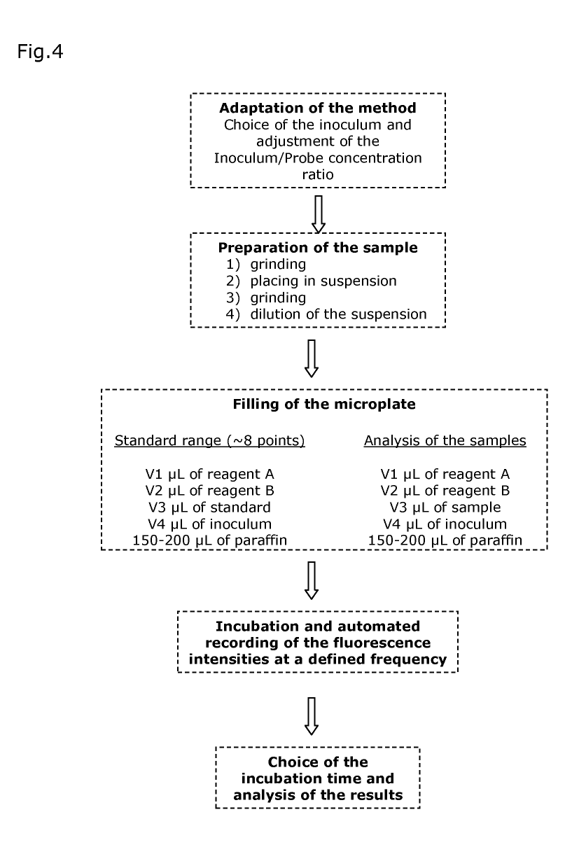

FIG. 4 shows diagrammatically the implementation of the method according to the invention under anaerobic conditions;

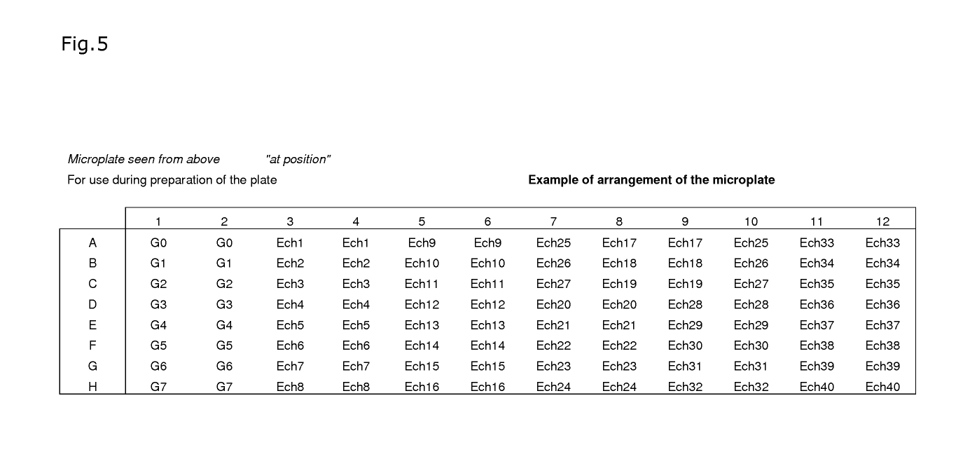

FIG. 5 gives an example of the arrangement of the samples in the wells of a microplate used according to Example 1;

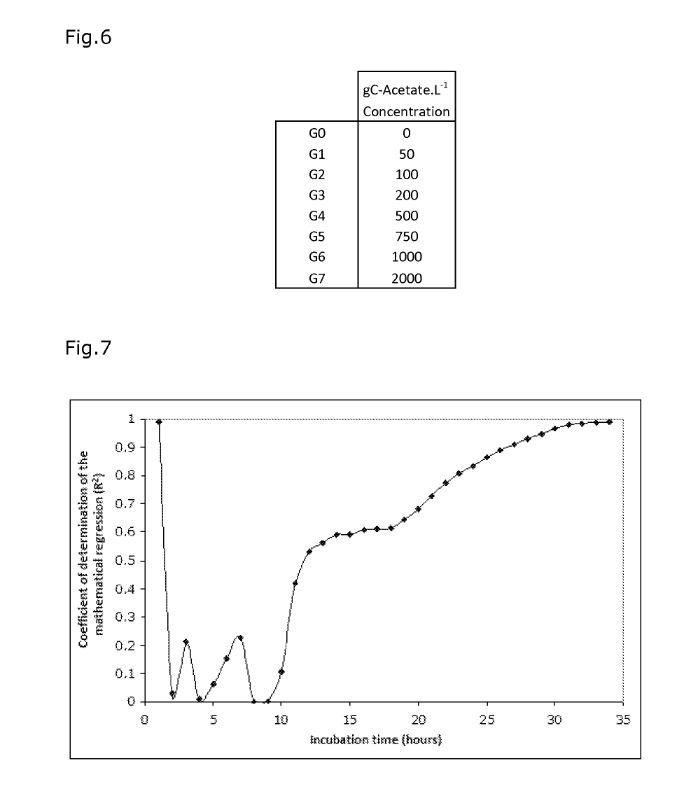

FIG. 6 gives an example of concentrations of standard range samples;

FIG. 7 is a graph showing the evolution of the coefficients determining the calibration curve as a function of the incubation time of the standard samples, under anaerobic conditions according to Example 1;

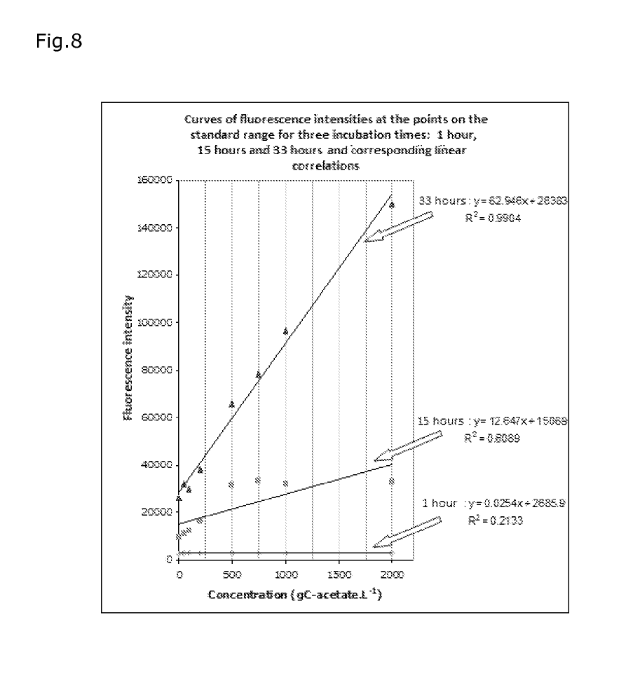

FIG. 8 gives three examples of calibration curves representing variations in the fluorescence intensity of the samples in the standard range as a function of the gC-Acetate.L⁻¹ concentration, for three incubation times: 1 hour, 20 hours and 33 hours under anaerobic conditions according to Example 1.

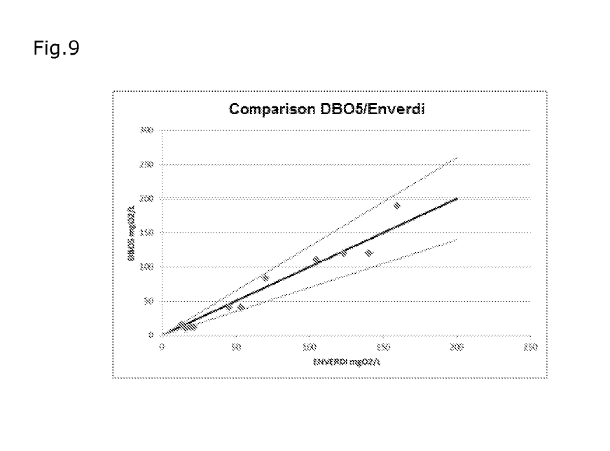

FIG. 9 illustrates the correlation between a conventional measurement of BOD₅ and the measurement carried out according to the invention with the Enverdi® kit according to Example 2. The black curve corresponds to the curve for which the correlation is equal to 1 (Enverdi® measurement=standardized BOD₅ measurement), the grey curves corresponding to the curve for which the correlation is equal to 1.3 (Enverdi® measurement=1.3 standardized BOD₅ measurement) and to the curve for which the correlation is equal to 0.7 (Enverdi® measurement=0.7 standardized BOD₅ measurement). (⋄) represents the measurements carried out on the samples according to Example 2.

The measurement support consists of a 96-well microplate with a transparent base. The reading is carried out preferably through the underside of the microplate. The benefit of this support is that it makes it possible to work on very small volumes of samples and reagents, and to carry out in parallel a large number of measurements on one or more samples.

The features of the plate vary according to the type of biodegradability to be measured. For aerobic biodegradability, the microplate is optionally covered by a culture film making it possible to allow the air to pass while avoiding evaporation. For anaerobic biodegradability, each well of the microplate is covered with paraffin, then the microplate is closed using a cover. Under anaerobic conditions with samples in suspension, the plate is sealed and can be turned over for reading from above so that the matter in suspension falls onto the paraffin and does not obstruct the measurement.

According to the type of measurement to be carried out, aerobic or anaerobic, a different protocol is implemented.

### Aerobic Biodegradability Measurement Protocol (for Example Measurement of BOD₅)

The steps of this protocol are summarized in FIG. 1.

Selecting the Inoculum

Before implementing the method, it is necessary to select the inoculum of microorganisms to be used to carry out the measurement.

For aerobic biodegradability measurements, the choice of inoculum depends on:

the availability or not at the site where the sample is collected, of an effluent of the wastewater treatment plant outlet water type with a high level of bacteria, having a composition that is sufficiently stable over time, of the outlet water from a wastewater treatment plant type or any other industrial effluent with a high level of bacteria;

the possibility of preparing an inoculum in situ from lyophilized strains, depending in particular on the availability of laboratory equipment, and the possibility of storage under the required conditions.

The inocula which can be used are:

outlet water from a wastewater treatment plant without tertiary disinfection treatment;

inlet water to a wastewater treatment plant, diluted between 10 and 100 times;

specific BOD₅ inoculum in tablets or lyophilized gelatin capsules;

biofilms suspension;

other mixture of bacteria or bacterial strains suitable for the application.

The wide choice of inocula that can be used for implementing the method according to the invention allows the method to be adapted to multiple applications in the field or in the laboratory. A person skilled in the art will be able to choose the suitable inoculum in light of his knowledge.

Characterization of the Inoculum

A preliminary phase of study and characterization of the inoculum is necessary in order to determine the association between the development of the intensity of absorbance-fluorescence and the industrial parameter that one wishes to express. This step consists of analyzing in parallel under the operating conditions of the invention and according to the reference method with the same inoculum:
either synthetic solutions containing increasing concentrations of a substrate (for example a mixture of glucose-glutamic acid, cellulose, sodium acetate etc.)
or preparations of a sample at different dilutions
or preparations of several samples of different matrices

Comparison of the intensity profiles or the fluorescence development rate profiles with the BOD₅ measurement will serve to associate the standard range with the industrial parameter, and therefore to quantify the samples analyzed according to the method of the invention, for this inoculum.

Selecting the Incubation Time

After the choice of one or more inocula, a second phase of adapting the implementation of the method according to the invention consists of determining the incubation time.

Two types of analyses are carried out in parallel on real samples and synthetic control solutions in increasing concentrations corresponding to the calibration range:
measurements according to the method of the invention; in this case, an analysis is carried out every 30 minutes, over a total incubation period of 24 h.
measurements of the industrial parameter, such as for example a standard BOD₅ measurement.

Then, a comparison is carried out between the results obtained for each type of measurement. For each incubation time in the method according to the invention, the quality of the calibration curve, i.e. either the value of the coefficient of determination of the calibration curve or the measured maximum instantaneous rates, and the calculated concentrations of the samples and control points are recorded. The comparison of the results obtained for each incubation time with the reference method makes it possible to determine the incubation time at which both a good correlation of the standard range and a good prediction of the industrial parameter of the samples and control points can be obtained. For example, the incubation time chosen is that at which the coefficient of determination of the calibration curve is equal to 0.98 and the standard prediction error between a potential value measured according to a standardized method and a value measured according to the method is less than 30%.

If several inocula have been assayed, the one giving the best results in the shortest incubation time is selected.

Sample Preparation Protocol

The samples to be analyzed are subjected to simple homogenization before sampling, and dilutions as a function of their concentration and ranges used.

The table presented in FIG. 2 shows the dilutions of the “typical” samples as a function of the range used, for information purposes. Other ranges can be developed according to the needs of users.

The sample is diluted in distilled water or in a buffer, an example of the composition of which is given in the table in FIG. 3.

Microplate Filling Protocol

The wells of a microplate with a transparent base are filled according to the following protocol:

V₁ μL of reagent

V₂ μL of inoculum

V₃ μL of sample, standard solution or control solution.

Optimization of the volumes and concentrations of reagents makes it possible to increase the sensitivity of the method, to reduce the reading and incubation time and improve the correspondence with the standard methods.

### Reagent

The reagent is a solution of a fluorescent and/or colorimetric bioreagent that is an indicator of microbial proliferation, diluted in a buffer.

A fluorescent probe used is for example a fluorescent redox indicator sensitive to the catabolism of the organic matter in the sample. The fluorescent bioreagent can be chosen in particular from the group constituted by the commercial compositions of resazurin derivatives (marketed in particular under the product names Alamarblue™, see U.S. Pat. No. 5,501,959, or MU, or PrestoBlue™). Alamarblue™ has the double advantage of changing colour and also forming a fluorescent product. The colour change between the blue non-fluorescent oxidized form and the violet reduced fluorescent form is marked and can be seen with the naked eye. The chosen fluorescent reagent is used for its sensitivity and for its ability to reveal all metabolisms (aerobic or anaerobic).

The dilution buffer is chosen so as not to interfere with the measurement. It can be prepared according to the protocol presented in the table in FIG. 3.

The dilution rate of the fluorescent and/or colorimetric bioreagent in the buffer is defined as a function of the applications and the measurement ranges.

According to a particular embodiment of the method according to the invention, the pH of solution 1 can be adjusted, within a range varying between 6.5 and 8.5.

According to another embodiment according to the invention, the bioreagent is only diluted in solution 1 (FIG. 3).

The volume V₁ of diluted reagent 1 is comprised between 10 and 180 μL, advantageously comprised between 50 and 150 μL, and preferably equal to 90 μL.

### Inoculum

The inoculum, selected in the first step, can be a “real” sample, for example, wastewater treatment plant inlet or outlet water, diluted or not with distilled water or in a buffer such as that described in the table in FIG. 3. It can also be a “synthetic” inoculum in the form of tablets or gelatin capsules of lyophilized bacteria.

The volume V₂ of inoculum is comprised between 10 and 180 μL, advantageously comprised between 50 and 150 μL, and preferably equal to 90 μL.

### Sample/Standard/Control Solution

The volume of the solution to be analyzed which is either a sample, diluted or not, or a point of the standard range, or a control solution, is comprised between 10 and 180 μL, advantageously comprised between 50 and 150 μL, and preferably equal to 90 μL.

Incubation

The incubation is carried out at a temperature that is adapted according to the potential to be measured. For measurements of BOD₅, the incubation is carried out at a temperature comprised between 29° C. and 31° C., preferably equal to 30° C. The total incubation period is comprised between 1 hour and 24 hours, with absorbance—fluorescence measurements at a defined frequency. The frequency of the measurements is at most every 15 minutes and at least hourly. The incubation is carried out directly in the reader which controls the temperature and agitation conditions.

This protocol makes it possible to automatically acquire absorbance—fluorescence readings at very regular time intervals, in order to continuously monitor the biodegradation.

Analysis of the Results According to Method 1

The analysis of the results is carried out in three steps:

Step 1: Preparing the Method of Calculation Between Fluorescence Intensity and mgO₂.L⁻¹Concentration.

Three cases can be distinguished:

Case 1: A complete standard range measurement is carried out in parallel with the measurements of the samples having unknown concentrations to be analyzed in order to obtain a calibration curve. For each sample, the equation of the calibration curve representing the fluorescence intensity as a function of the mgO₂.L⁻¹ concentration is used in order to calculate the concentrations of the samples analyzed. This calibration curve is obtained by previously defining a proportionality relationship between a glucose-glutamic acid concentration and a BOD₅ value expressed as mgO₂.L⁻¹ by analysis of synthetic glucose-glutamic acid solutions according to the standardized BOD₅ method.

Case 2: Only the samples having unknown concentrations are analyzed and an internal mathematical model is used to calculate their concentrations. The equation of the internal model associating the fluorescence intensity with the concentration of mgO₂.L⁻¹ is determined by utilizing results from a number of previous experiments, with a minimum number of four. For each sample, the internal model equation is used to determine the concentrations of the samples. Control solutions having known concentrations can optionally be analyzed in parallel in order to verify the validity of the model.

Case 3: The samples having unknown concentrations as well as control solutions having known concentrations are analyzed. The fluorescence intensity measurements on the control solutions are used as means of comparison in order to adjust the equation of the mathematical model used in the abovementioned case 2. The new equation obtained is used to determine the concentrations of the samples analyzed.

The dilution factor of the samples is taken into account in calculating the concentrations.

### Step 2: Verification of the Incubation Time

It is useful to verify, if possible, that the predetermined conditions are well adapted to the experiment, in particular when the inoculum chosen is a “real” inoculum, originating from a sample. Variations in the wastewater treatment plant process or climatic disturbances can cause alterations in the quantity and quality of the bacteria present in the sample, leading to incubation conditions that are different from the conditions originally chosen.

Verification of the incubation time can be done according to one of the following methods:

Monitoring of the coefficient of determination R² of the calibration curve over time: the variation in R² over time is compared with that obtained during the first experiments, allowing the incubation time to be determined. The value of R² at the chosen incubation time is also recorded. Its value must be greater than 0.98. This method is valid if a standard range is used in addition to the samples on the plate.

Verification of the fluorescence intensity of the control solutions: the fluorescence intensity values of the control solutions are compared with the expected values at the chosen incubation time. The observed deviation must be less than 10%. This method is valid if one or more control solutions have been used.

Verification of the concentrations of the control solutions: on the basis of the defined equation (calibration or internal model equation), the concentrations of the control solutions are calculated for each incubation time. The concentrations of the control solutions, calculated at the incubation time, are compared with the actual expected concentrations. The observed deviation must be less than 10%. This method is valid if one or more control solutions have been used.

If these verifications lead to the conclusion that the predefined incubation time is not the optimum time for this experiment, another incubation time must be chosen for reading the concentrations. This arbitrary choice is established based on the observations carried out on the calibration curve, in particular concerning the change in R² over time, as well as the change in the fluorescence intensities and/or the concentrations of the control solutions calculated over time.

### Step 3: Determining the Concentrations of the Samples

After reading the fluorescence intensity for each sample at the chosen incubation time, the concentration of an industrial parameter, in particular mgO₂.L⁻¹ for a measurement of BOD₅, is calculated according to the method of determination described in step 1.

Analysis of the Results According to Method 2

The analysis of the results is carried out in three steps:

Step 1: Preparing the Method of Calculation Between Fluorescence Intensity and mgO₂.L concentration.

Three cases can be distinguished:

Case 1: A complete standard range measurement is carried out in parallel with the measurements of the samples having unknown concentrations to be analyzed in order to obtain a calibration curve. The calibration curve associates the maximum rate of fluorescence change during the analysis with the concentration of the standard solution, which can be expressed directly with the industrial parameter unit, according to the results obtained during the characterization of the inoculum. For the samples, the maximum rate of fluorescence change is determined after a period of adaptation of the bacteria of the sample to the new medium (temperature, nutrients, pH etc) which can be from 1 h to 5 h.

Case 2: Only the samples having unknown concentrations are analyzed and an internal mathematical model is used to calculate their concentrations. The equation of the internal model associating the maximum rate fluorescence change with the content in mgO₂/L is determined by utilizing results from a number of previous experiments, with a minimum number of four. For each sample, the internal model equation is used to determine the concentrations of the samples. Control solutions having known concentrations can optionally be analyzed in parallel in order to verify the validity of the model.

Case 3: The samples having unknown concentrations as well as control solutions having known concentrations are analyzed. The fluorescence intensity measurements on the control solutions are used as means of comparison in order to adjust the equation of the mathematical model used in the abovementioned case 2. The new equation obtained is used to determine the concentrations of the samples analyzed.

### Step 2: Determining the Concentrations of the Samples

After reading the fluorescence intensity for each sample and processing the data in order to determine the maximum rate of fluorescence change during the analysis, the concentration as an industrial parameter, in particular in mgO₂/L for a measurement of BOD₅, is calculated according to the determination method described in step 1.

### Anaerobic Biodegradability Measurement Protocol (for Example Measurement of BMP)

This protocol is summarized in FIG. 4.

Selecting the Inoculum

For anaerobic biodegradability measurements, the choice of inoculum depends on:

the availability or not at the sampling site, of sludges from digesters or from an anaerobic bacterial system having a composition that is sufficiently stable over time;

the possibility of preparing a model inoculum starting from cultures of strains, a cocktail of strains, a laboratory digester, or lyophilized strains.

The inocula which can be used are:

sludges from digesters

any biomass originating from an anaerobic system

suspensions of aerobic biofilms and/or anaerobic biofilms

bacterial cultures, or lyophilized bacteria

another mixture of bacteria or bacterial strains suitable for the application.

After choosing the inoculum, it is important to adjust the bacteria/fluorescent bioreagent concentration ratio. This is carried out in an empirical manner by carrying out the assay for different concentrations and/or volumes of inoculum and different assays of concentrations on the bioreagent. These assays are carried out at the calibration points, thus allowing the ratio to be adjusted in order to obtain valid absorbance—fluorescence intensities. The total volume in a well cannot exceed 300 μL.

According to a particular embodiment of the method according to the invention, measurements can be carried out in parallel on samples with known BMPs in order to verify the accuracy of the results obtained according to the invention.

Characterization of the Inoculum

A preliminary phase of study and characterization of the inoculum is necessary in order to determine the association between the change in the intensity of absorbance—fluorescence and the industrial parameter BMP that it is desired to express. This step consists of analyzing in parallel under the operating conditions of the invention and according to the BMP reference method with the same inoculum:

either synthetic solutions containing increasing concentrations of a substrate (for example, sodium acetate, cellulose, etc.)

or preparations of a sample at different dilutions (example: a suspension of a sample of biodegradable waste or sewage sludges at different concentrations)

or preparations of several samples of different matrices (e.g.: biodegradable waste, agri-food waste, sewage sludges, slurries etc.).

Comparison of the intensity profiles or the fluorescence rate of change profiles with the industrial parameters obtained by the reference method will serve to associate the standard ranges with the industrial parameters, and therefore to quantify the samples analyzed according to the method of the invention, for this inoculum.

Sample Preparation Protocol

The method according to the invention allows the analysis of samples in the liquid state and samples in the solid state.

The samples to be analyzed are taken in such a way that the sample is representative of entire body of matter.

Each sample is then mixed using a food blender.

The sample is then placed in suspension. For example, 20 g of solid sample is suspended in distilled water to make up 200 g. This mass is adapted according to how easily the suspension forms, the availability or the homogeneity of the product.

If it is desired to compare the result of the analyses according to the method of the invention with BMPs, in this step it is desirable to measure the density of the samples, if the state of the sample allows.

The suspension obtained is again homogenized by grinding, with a food blender.

This suspension or the raw effluent can then be diluted in 5 concentrations: 1/50, 1/100, 1/200, 1/250, 1/500 are conventionally-used dilutions.

The measurement can thus be used on the raw sample or the “stock” suspension, as well as on the whole or a selection of diluted solutions.

Microplate Filling Protocol

FIG. 5 gives an example of a microplate arrangement under anaerobic conditions. The standard solutions G0 to G7 correspond to different increasing standard range concentrations, examples of the values of which are given in FIG. 6. Information on the microplate organization diagram is entered in the absorbance—fluorescence reader program, in terms of identification of the samples.

The wells of a microplate with a transparent base are filled according to the following protocol:

V₁ μL of reagent A

V₂ μL of reagent B

V₃ μL of sample, standard solution or control solution

V₄ μL of inoculum

150-200 μL of paraffin

### Reagent A

Reagent A is a buffer solution, an example composition of which is given in the table in FIG. 3. In this example, the pH of solution 1 is adjusted to the value 7.2.

According to a particular embodiment of the method according to the invention, the pH of solution 1 can also be adjusted within a range varying for example between 6.5 and 8.5, or within another pH range suited to the application.

According to another embodiment of the method according to the invention, the bioreagent is only diluted in solution 1.

### Reagent B

Reagent B is a fluorescent and/or colorimetric bioreagent that is an indicator of microbial proliferation, diluted in a buffer or not.

The dilution rate of reagent B in a buffer is comprised between 1 and 10. For example, for questions of sensitivity, reagent B can be used at the concentration as marketed, with a volume of 100 μL.

According to a particular embodiment of the method according to the invention, the volume of the pure product can also be slightly increased, or the product can be diluted, from 50 μL à 150 μL.

### Raw Sample, Diluted or Not/Standard/Control Solution

A volume of 100 μL of sample is introduced into each well.

### Inoculum

After sampling, the inoculum is filtered using 1.2 μm mesh PES (polyethersulphone) filters, so as to retain the coarser particles of organic matter while allowing the bacterial cells constituting the inoculum to pass.

According to a particular embodiment of the method according to the invention, in a concentrated or stressed culture medium, a finer filtration mesh can be used. This choice can be a means of selecting bacterial communities or bacterial strains favourable to analysis or prediction.

According to another embodiment, if the inoculum has a low organic matter content, filtration can be avoided.

Following this first filtration phase, the inoculum is introduced diluted or not into a well, conforming to a total reagent volume comprised between 280 μL and 300 μL.

For example, an inoculum originating from a digester treating water purification by-products is introduced into the well in a volume of 30 μL.

### Paraffin

So that the measurement can take place under anaerobic conditions, a drop of paraffin is deposited on the liquid reagent of each well.

According to a preferred embodiment of the method according to the invention, a volume of 150 μL to 200 μL paraffin is deposited on the surface of each well.

Incubation

Throughout the incubation period, the fluorescence intensities are collected automatically at a determined frequency, for example hourly. At each measurement time, the fluorescence intensity at the calibration points makes it possible to plot a calibration curve representing the correlation between fluorescence intensity and the acetate concentration at the normal points or fluorescence intensity and BMP at the calibration points. The incubation takes place directly in the reader which controls the temperature and stirring conditions.

Analysis of the Results

### Step 1: Selecting the Incubation Time

Under anaerobic conditions, the selection of the incubation time is carried out automatically, at the same time as the measurement of the samples, by means of the data processing algorithm.

The parameter monitored for selecting the time of analysis of the fluorescence intensities is the coefficient of determination R² of the calibration curve corresponding to the standard solutions G0 to G7, obtained for each measurement time.

At the end of the incubation, the data analysis time is chosen for the calibration curve having the best coefficient of determination. A verification of the fluorescence intensity kinetics over time for each sample or standard solution is carried out and makes it possible to discard undesirable values. For example, a reduction in the fluorescence intensity, a value of the fluorescence intensity of a sample greater than the value of the fluorescence intensity of the highest point of the standard range, can reflect fluorescence intensities associated with a fermentary, and not a methanogenic, metabolism.

### Step 2: Method for Calculating the Methane Potential

Once the analysis time has been selected, the fluorescence intensities of the samples having unknown concentrations are converted to gC-Acetate.Kg⁻¹ using the calibration curve.

Reference is then made to a database constituted by samples having known gC-Acetate.Kg⁻¹ and BMP LCH₄.Kg⁻¹ raw matter values. This database makes it possible to carry out the prediction for samples the methane potential of which is to be predicted in terms of LCH₄.kg⁻¹ raw matter. Controls can be carried out in parallel for verification purposes on samples having known BMPs.

According to a particular embodiment of the method according to the invention, the methane potential of the unknown samples is estimated directly based on the calibration curve and expressed in LCH₄.kg⁻¹ raw matter. This is possible in the case where standard values alone allow a good prediction of the methane potential of complex samples, i.e. each calibration point is characterized by a methane potential.

Other features and advantages of the invention will become apparent on reading the detailed description of the example below.

### EXAMPLE 1

### Prediction of the Methane Potential Under Anaerobic Conditions in Digester Sludges

In this example, the inoculum is constituted by slurries from a “continuously stirred tank reactor” digester of a sewage treatment plant.

The first step consists of collecting the inoculum and the samples.

The digester sludges are collected from the mid-height of a “continuously stirred tank reactor”. Inlet samples are also collected.

The inoculum is stored in an incubator at 35° C., 55° C. or at the temperature of the digester.

The samples are then prepared according to the anaerobic biodegradability measurement protocol described in the invention.

The solid samples that cannot be taken with a 5 mL pro pipette are subjected to the following preparation steps:
grinding
placing in suspension
grinding
dilution

The so-called liquid samples that are capable of being taken with a 5 mL pro pipette are subjected to two preparation steps only:
grinding
dilution

The density of the samples is then measured.

The subsequent steps are filling the microplate and preparing the inoculum.

The filling of the microplate is defined by the technician, who arranges the standard solutions and the samples according to a personal arrangement. Information on this arrangement is entered in the fluorescence reader program. An example arrangement is shown diagrammatically in FIG. 5, G0 to G7 representing the standard range solutions.

50 μL of buffer is placed in each well of the microplate with a transparent base corresponding to one analysis. If the 96 wells are used, then the buffer is distributed into all of the wells. If only a few wells are necessary, then a distribution is made into these wells only, the remaining wells being available for use for future analyses.

100 μL of reagent B is introduced into each of the selected wells.

Then, according to the previously-agreed arrangement, 100 μL of each of the standard solutions G0 to G7 supplied is arranged in the corresponding wells, and 100 μL of sample (raw, diluted or not) is distributed into each of the corresponding wells on the surface of the microplate.

Using a syringe and a syringe filter, the inoculum is passed through a 1.2 μm filter. Then 30 μL of this filtered inoculum is distributed into each of the wells in order to complete the reaction mixture.

In order to homogenize the mixture and to dislodge droplets previously retained on the edges of the wells, the microplate is manually tapped against the work surface.

The paraffin is allowed to melt, then drawn up using a 1 mL pro pipette. A volume of 150 to 200 μL of paraffin is deposited on the surface of the reagent in each of the wells. The microplate is then closed using a cover in order to ensure anaerobic conditions.

The microplate thus filled is placed in the reader for incubation and measurement. The fluorescence intensities are collected automatically every hour. At each collection, the fluorescence intensity at the standard points G0 to G7 makes it possible to draw a calibration curve representing the linear correlation between the fluorescence intensity and the acetate concentration at the normal points. The coefficient of determination of the linear correlation makes it possible to reveal the correspondence between the observed data and the model. FIG. 7 shows the evolution of the coefficients of determination of the calibration curve as a function of the incubation time of the standard samples. Three examples of calibration curves representing the variations in the fluorescence intensity of the samples of the standard range as a function of the gC-Acetate.L⁻¹ concentration are given in FIG. 8 for incubation times of 1 hour, 20 hours and 33 hours. It is noted that the coefficient of determination R² of the calibration curve improves over time. For an incubation time equal to 33 hours, R² is equal to 0.99.

An incubation time of 33 hours is therefore chosen automatically for analyzing the fluorescence intensities of the samples.

Then, the fluorescence intensities of the samples having unknown concentrations are converted to gC-Acetate.L⁻¹ using the calibration curve equation (FIG. 8). Then reference is made to a database constituted by samples with known gC-Acetate.L⁻¹ and BMP LCH₄.L⁻¹ raw matter values. This database makes it possible to carry out the prediction for samples of the methane potential of which is to be predicted in terms of LCH₄.kg⁻¹ raw matter.

Of course, the invention is not limited to the example which has just been described and numerous adjustments can be made to this example without exceeding the scope of the invention.

The method according to the invention allows BOD₅ equivalents and BMP equivalents to be obtained in less than 24 hours and in less than 35 hours respectively with an excellent correlation compared with standard methods for measuring these two industrial parameters.

### EXAMPLE 2

### Prediction of BOD₅ in Samples Originating from an Urban Wastewater Treatment Plant

The samples are samples carried out throughout the process in an urban wastewater treatment plant.

The duration of the incubation in the presence of the fluorescent bioreagent is 15 h at 30° C. on a plate covered with an aerobic film;

The water at the inlet of the wastewater treatment plant diluted 25 times is used as an inoculum type (microorganisms)

The incubation mixture comprises 90 μL of reagent comprising the fluorescent probe, 90 μL of inoculum and 90 μL of sample to be tested.

Reference is made to a glucose/glutamic acid range of 0 to 250 mg/L.

The reading is taken through the underside in a spectrophotometer at the following wavelengths: excitation 540 nm; emission 600 nm.

In parallel, the BOD₅ of the samples is measured by a conventional standardized method.

The results are given in FIG. 9.

All the measurements are comprised between −30 and +30% with respect to the straight line the correlation coefficient of which is equal to 1.

The measurement according to the invention therefore makes it possible to obtain a very good evaluation of the quantity of organic matter present very rapidly (15 hours).

1. Method for the direct measurement of the biodegradability of organic samples comprising the following steps:
preparation of the sample,
incubation of the sample for a duration comprised between 1 and 48 hours, advantageously between 12 and 24 hours in a microplate, with a fluorescent and/or colorimetric bioreagent and an inoculum of micro-organisms capable of degrading said sample, said microorganisms being optionally prepared from lyophilized strains or from a culture of bacterial strains
analysis of the absorbance—fluorescence emitted by the mixture over time, said analysis of absorbance—fluorescence comprising the following two steps:
a) measurement of an intensity of absorbance—fluorescence emitted following degradation of the sample by the inoculum of micro-organisms, said fluorescence intensity profile obtained allowing:
either determination of the minimum measurement time by analysis of a coefficient of determination of a calibration curve, said calibration curve associating the intensity of the fluorescence with the concentration of organic matter being obtained by carrying out measurements of absorbance—fluorescence on samples of known increasing concentrations constituting a standard range, and/or by comparison of the results obtained on samples of known concentrations by a usual “standardized” method,
or deducing instantaneous rate of biodegradation profiles,
b) use of the intensity of absorbance—fluorescence measurement for calculating a reference organic matter concentration, using a correlation either with a standard range, or with a mathematical model, said method comprising moreover a step of calculating an industrial parameter from the organic matter concentration calculated in step b).

2. Method according to claim 1, characterized in that measuring the absorbance—fluorescence intensity is carried out through the underside of the plate.

3. Method according to claim 1, characterized in that the sample to be analyzed is collected at a treatment site and in that the inoculum of microorganisms originates from a bacterial system present on this same site, having a composition that is stable over time, said inoculum optionally being passed through 1.2 μm mesh PES filters.

4. Method according to claim 1, characterized in that the measurement is carried out, advantageously at least hourly and at most every 15 minutes, under aerobic conditions, either leaving the microplate open, or covering it with a film allowing oxygen exchange without evaporation.

5. Method according to claim 1, characterized in that the measured fluorescence is converted to mgO₂.L⁻¹, the unit in which BOD₅ is expressed according to the standard, by comparison with a standard range or by use of a mathematical model associating the fluorescence intensity with the concentration.

6. Method according to claim 5, characterized in that the mathematical equation is adjusted as a function of the fluorescence intensities measured on control solutions having known concentrations placed under the same conditions as the samples to be analyzed.

7. Method according to claim 1, characterized in that the measurement is carried out under anaerobic conditions, in particular by covering each well of the microplate with paraffin, then closing the microplate using a cover, the plate optionally being turned over so that the fluorescence measurement takes place from above.

8. Method according to claim 7, characterized in that the measurement time, chosen automatically, is that at which the coefficient of determination of the calibration curve is the closest to 1 over a total incubation period.

9. Method according to claim 7, characterized in that the fluorescence emitted is converted to LCH4.kg⁻¹ raw matter, in particular according to the BMP standard (methane potential) according to a calculation comprising the following steps:
converting the profiles of absorbance-fluorescence intensities or the profiles of instantaneous rates to gC-Acetate.Kg⁻¹ using the mathematical equation originating from the linear regression of the calibration curve, or
referring to a database constituted by samples with known gC-Acetate.Kg⁻¹ and BMP LCH₄.Kg⁻¹ raw matter values in order to predict the methane potential in terms of LCH4.kg⁻¹ raw matter.

10. Method according to claim 9, characterized in that the methane potential of unknown samples is directly estimated from the calibration curve in terms of LCH4.kg⁻¹ raw matter.

11. Method according to claim 1, characterized in that it utilizes a kit comprising at least a microplate, at least a fluorescent and/or colorimetric bioreagent and standard and/or control solutions.

12. Method according to claim 1, characterized in that the step for analyzing the absorbance-fluorescence is implemented by a system comprising a fluorescence reader suited to the microplate format and calculation means arranged for implementing said method.

13. Method according to claim 8, characterized in that the fluorescence emitted is converted to LCH4.kg⁻¹ raw matter, in particular according to the BMP standard (methane potential) according to a calculation comprising the following steps:
converting the profiles of absorbance-fluorescence intensities or the profiles of instantaneous rates to gC-Acetate.Kg⁻¹ using the mathematical equation originating from the linear regression of the calibration curve, or
referring to a database constituted by samples with known gC-Acetate.Kg⁻¹ and BMP LCH₄.Kg⁻¹ raw matter values in order to predict the methane potential in terms of LCH4.kg⁻¹ raw matter.
